(19) United States
(12) Patent Application Publication
(10) Pub. No.: US 2005/0026862 A1
(43) Pub. Date: Feb. 3, 2005

(54) Cosmetic or dermatological preparation

(75) Inventors: Thomas Blatt, Wedel (DE); Franz Stab, Echem (DE); Rainer Wolber, Hamburg (DE); Ludger Kolbe, Dohren (DE); Stefan Gallinat, Wedel (DE); Ute Breitenbach, Hamburg (DE); Kyra Sanger, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/896,786

(22) Filed: Jul. 21, 2004

Related U.S. Application Data
(63) Continuation of application No. PCT/EP03/00017, filed on Jan. 3, 2003.

(30) Foreign Application Priority Data
Jan. 21, 2002 (DE) 102 02 251.8

(51) Int. Cl. A61K 48/00; C12Q 1/68

(52) U.S. Cl. 514/44; 514/267; 435/6

(57) ABSTRACT

The invention is a cosmetic or dermatological preparation comprising at least one reaction product of DNA selected from the group consisting of oxidation products of mononucleosides, oxidation products of nucleobases, photoproducts of mononucleosides, and photoproducts of nucleobases. In one embodiment, the invention includes a cosmetic or dermatological preparation comprising at least one 8-hydroxypurine nucleoside. The invention also includes methods for preventing or treating degenerative or inflammatory skin symptoms, reducing skin wrinkles, preventing skin aging or firming the skin comprising applying the cosmetic or dermatological preparation to the skin, optionally in combination with laser or abrasive treatment.

### CROSS-REFERENCE TO RELATED APPLICATIONS

This is a continuation application of PCT/EP03/00017, filed Jan. 3, 2003, which is incorporated herein by reference in its entirety, and also claims the benefit of German Priority Application No. 102 02 251.8, filed Jan. 21, 2002.

### FILED OF THE INVENTION

The present invention relates to cosmetic or dermatological preparations which comprise particular products obtained from DNA as active ingredient.

### BACKGROUND OF THE INVENTION

The human skin is the largest human organ and performs a number of vital functions. Having an average surface area of about 2 m² in adults, it has a prominent role as a protective and sensory organ. The purpose of this organ is to transmit and avert mechanical, thermal, chemical and biological stimuli. In addition, it has an important role as a regulatory and target organ in human metabolism.

Cosmetic skin care is primarily to be understood as meaning the strengthening or restoring of the natural function of the skin as a barrier against environmental influences (e.g. dirt, chemicals, microorganisms) and against the loss of endogenous substances (e.g. water, natural fats, electrolytes), and also the assistance of its horny layer in its natural regeneration ability in cases of existing damage.

Impairment of the barrier properties of the skin may lead to increased absorption of toxic or allergenic substances or to attack by microorganisms and consequently to toxic or allergic skin reactions.

Another aim of skincare is to compensate for the loss by the skin of sebum and water caused by daily washing. This is particularly important if the natural regeneration ability is inadequate. Furthermore, skincare products should protect against environmental influences, in particular against sun and wind, and delay skin aging.

It is known that external stress in a cell, for example as a result of UV light, oxidative stress, free radicals or mechanical irritation, displays a multitude of effects. One of these effects is damage to the DNA endogenous to the cell. This produces various reaction products, such as DNA fragments of varying size, photochemical reaction products such as [2+2]-cycloaddition products and other photodimers, and oxidation products of individual nucleotides or nucleosides. These stimulate various repair mechanisms within the cell, as a result of which the cell repairs the resulting damage by restoring the defective DNA, removing the fragments, the cell divides or dies if the damage cannot be repaired. This stimulation of the cellular repair mechanisms results, in the case of damaged or stressed tissue overall, in a reaction which counteracts damage or stress.

To stimulate the skin's own regeneration operations, an extremely wide variety of cosmetic preparations is obtainable which in most cases comprise vitamins, provitamins, various plant extracts and so on as active ingredient.

### BRIEF SUMMARY OF THE INVENTION

Accordingly, it is the subject matter of this invention to use the reaction products of DNA, such as, for example, photodimers, other photoproducts or oxidation products of the nucleotides, nucleosides or nucleobases which also arise in the cell as cosmetic active ingredients. In this connection, 8-hydroxypurines, such as 8-hydroxyguanosine (CAS No. 3868-31-3), a reaction product formed from DNA under oxidative stress: 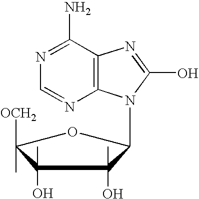
thymidine photodimers: 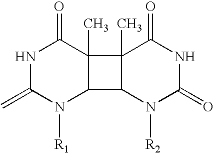
which are formed from DNA by photo-induced {2+2]-cycloaddition of, for example, two adjacent pyrimidine bases, where R₁ and R₂, independently of one another may either be carbohydrates or derivatives thereof, such as phosphorylated carbohydrates or else nucleotide chains, or else various binding sites of the DNA which is itself otherwise unchanged or differently changed at another site, analogously also other cyclobutane nucleotide dimers also of different nucleotide bases, and pyrimidine-pyrimidone-6,4-photoproducts: 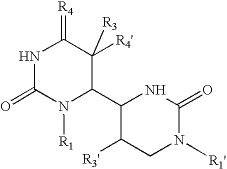
   where R₃ and R₃′, independently of one another, are methyl or hydrogen, and R₄ and R₄′, independently of one another, are the oxygen of a keto group or the double-bonded NH group in an imido group, and R₄′ in the photoreaction product is an amino or hydroxyl group, are of particular interest.

As treatment methods for psoriasis, vitiligo, as a method for preventing photo-induced skin aging, UV-induced dermatoses and as a method for reducing the susceptibility to skin cancer and UV-induced hyperproliferative disorders, the specification U.S. Pat. No. 5,955,059 discloses the application of an effective amount of DNA fragments with a length of from 2 to 200 bases, where the DNA fragments are single strands, double strands or mixtures thereof.

The British patent application 2,336,157 describes the use of single-strand DNA fragments, deoxynucleotides, dinucleotides and/or dimers of dinucleotides for the preparation of medicaments for stimulating the p53 activity in cells.

Although the aim of these two inventions remains unclear, these specifications were in any case unable to point the way to the present invention. In particular, nothing concerning oxidative or photochemical reaction products of individual nucleobases or nucleosides is disclosed.

It was thus an object of the present invention to find novel cosmetic and/or dermatological active ingredients which simulate oxidative or photochemical stress in cells and in so doing stimulate the cell's own repair mechanisms.

### DETAILED DESCRIPTION OF THE PREFERRED EMBODIMENTS

It has been found, in a manner which could not have been predicted by the person skilled in the art, that cosmetic or dermatological preparations comprising oxidation products or photoproducts of mononucleosides or nucleobases remedy the disadvantages of the prior art. In this connection, it is preferred when oxidation products of mononucleosides or photoproducts of nucleobases are used. It is particularly preferred when the oxidation products of mononucleosides used are 8-hydroxypurine nucleosides, or the photoproducts of nucleobases used are pyrimidine-6,4 photoproducts.

It is very particularly preferred when the 8-hydroxypurine nucleoside used is 8-hydroxyguanosine.

In this connection, it is preferred when the concentration of oxidation products and/or photoproducts of mononucleosides and/or nucleobases is 0.000001 to 0.1% by weight, particularly preferably 0.000001 to 0.001% by weight.

The cosmetic or dermatological preparations according to the invention are preferably used for the prevention or treatment of degenerative or inflammatory skin symptoms, for the care of the skin or aging skin, and also for preventing skin aging or for firming the skin.

The cosmetic or dermatological preparations according to the invention are extremely satisfactory preparations in every respect. It had not been foreseen by the person skilled in the art that the preparations according to the invention
better maintain or restore the barrier properties of the skin,
better counteract the skin from drying out,
have a better effect against skin aging and
better protect the skin against environmental influences than the preparations of the prior art.

Use of cosmetic or topical dermatological preparations with an effective content of active ingredient used according to the invention surprisingly enables effective treatment, but also prophylaxis
of deficient, sensitive or hypoactive skin states or deficient, sensitive or hypoactive states of skin appendages,
of signs of premature aging of the skin (e.g. wrinkles, age spots, telangiectases) and of the skin appendages,
of environmentally induced changes in the skin and the skin appendages (smoking, smog, reactive oxygen species, free radicals) and in particular photo-induced negative changes,
of photo-induced skin damage,
of circulatory disorders,
of itching,
of dry skin conditions and disorders of the horny layer barrier,
of stress sensitivity of the skin,
of inflammatory skin conditions, and atopic eczema, seborrheic eczema, polymorphous photodermatosis, psoriasis, vitiligo.
   The active ingredients according to the invention, or cosmetic or topical dermatological preparations with an effective content of active ingredient according to the invention, however, also surprisingly serves
to calm sensitive or irritated skin
to stimulate intracellular DNA synthesis, in particular in cases of deficient or hypoactive skin states
to increase cell renewal and regeneration of the skin
to increase the skin's own protective and repair mechanisms (for example for dysfunctional enzymes, DNA, lipids, proteins)
for pre- and post-treatment in cases of topical application of laser and abrasive treatments, which serve, for example, to reduce skin wrinkles and scars, in order to counteract the resulting skin irritations and to promote the regeneration processes in the damaged skin.

It is of advantage if the cosmetic or dermatological preparations according to the invention are emulsions. Emulsions are generally understood as meaning heterogeneous systems which consist of two liquids which are immiscible or miscible only to a limited degree with one another, and which are usually referred to as phases. In an emulsion, one of the two liquids is dispersed in the other liquid in the form of very fine droplets.

If the two liquids are water and oil and oil droplets are in finely distributed form in water, then this is an oil-in-water emulsion (O/W emulsion, e.g. milk). The basic character of an O/W emulsion is defined by the water. In the case of a water-in-oil emulsion (W/O emulsion, e.g. butter) the principle is reversed, the basic character here being determined by the oil.

Within the scope of the present disclosure, the expression “lipids” is sometimes used as the generic term for fats, oils, waxes and the like, as is entirely customary to the person skilled in the art. The terms “oil phase” and “lipid phase” are also used synonymously.

The aqueous phase of the preparations according to the invention optionally advantageously comprises alcohols, diols or polyols of low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols of low carbon number, e.g. ethanol, isopropanol, 1,2-propanediol, glycerol, and, in particular, one or more thickeners which can advantageously be chosen from the group consisting of silicon dioxide and aluminum silicates.

Preparations according to the invention in the form of emulsions advantageously comprise, in particular, one or more hydrocolloids. These hydrocolloids can advantageously be chosen from the group of gums, polysaccharides, cellulose derivatives, phyllosilicates, polyacrylates and other polymers.

The gums include saps from plants or trees which harden in the air and form resins, or extracts from aquatic plants. From this group, for the purposes of the present invention, gum arabic, carob flour, tragacanth, karaya, guar gum, pectin, gellan gum, carrageen, agar, algins, chondrus, xanthan gum, for example, can be chosen advantageously.

Also advantageous is the use of derivatized gums, such as, for example, hydroxypropyl guar (Jaguar® HP 8).

The polysaccharides and polysaccharide derivatives include, for example, hyaluronic acid, chitin and chitosan, chondroitin sulfates, starch and starch derivatives.

The cellulose derivatives include, for example, methylcellulose, carboxymethylcellulose, hydroxyethylcellulose, hydroxypropylmethylcellulose.

The phyllosilicates include naturally occurring and synthetic clay earths, such as, for example, montmorillonite, bentonite, hectorite, laponite, magnesium aluminum silicates such as Veegum®). These can be used as such or in modified form, such as, for example, stearylalkonium hectorite.

In addition, silica gels can also be used advantageously.

The polyacrylates include, for example, Carbopol grades from Goodrich (Carbopol 980, 981,1382, 5984, 2984, EDT 2001 or Pemulen TR2).

The polymers include, for example, polyacrylamides (Seppigel 305), polyvinyl alcohols, PVP, PVPNA copolymers, polyglycols.

The lipid phase of the cosmetic or dermatological emulsions according to the invention can advantageously be chosen from the following group of substances:
mineral oils, mineral waxes;
oils, such as triglycerides of capric or of caprylic acid, and also natural oils such as, for example, castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils, such as dimethylpolysiloxanes, diethylpolysiloxanes, diphenylpolysiloxanes
   and mixed forms thereof.

The oil phase of the emulsions of the present invention is advantageously chosen from the group of esters of saturated or unsaturated, branched or unbranched alkanecarboxylic acids having a chain length of from 3 to 30 carbon atoms and saturated or unsaturated, branched or unbranched alcohols having a chain length of from 3 to 30 carbon atoms, from the group of esters of aromatic carboxylic acids and saturated or unsaturated, branched or unbranched alcohols having a chain length of from 3 to 30 carbon atoms. Such ester oils can then advantageously be chosen from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, e.g. jojoba oil.

In addition, the oil phase can advantageously be chosen from the group of branched and unbranched hydrocarbons and hydrocarbon waxes, of silicone oils, of dialkyl ethers, the group of saturated or unsaturated, branched or unbranched alcohols, and the fatty acid triglycerides, namely the triglycerol esters of saturated or unsaturated, branched or unbranched alkanecarboxylic acids having a chain length of from 8 to 24, in particular 12-18 carbon atoms. The fatty acid triglycerides can, for example, advantageously be chosen from the group of synthetic, semisynthetic and natural oils, e.g. olive oil, sunflower oil, soybean oil, groundnut oil, rapeseed oil, almond oil, palm oil, coconut oil, palm kernel oil and the like.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric triglyceride, dicaprylyl ether.

Particularly advantageous mixtures are those of C₁₂₋₁₅-alkyl benzoate and 2-ethylhexyl isostearate, mixtures of C₁₂₋₁₅-alkyl benzoate and isotridecyl isononanoate, and mixtures of C₁₂₋₁₅-alkyl benzoate, 2-ethylhexyl isostearate and isotridecyl isononanoate.

Of the hydrocarbons, paraffin oil, squalane and squalene are to be used advantageously for the purposes of the present invention.

The oil phase can advantageously also have a content of cyclic or linear silicone oils, or consist entirely of such oils, although it is preferable to use an additional content of other oil phase components apart from the silicone oil or the silicone oils. Such silicones or silicone oils may be in the form of monomers, which are generally characterized by structural elements, as follows:

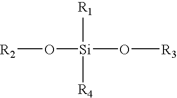

Linear silicones having two or more siloxyl units which are to be used advantageously according to the invention are generally characterized by structural elements, as follows: 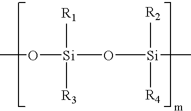
where the silicon atoms can be substituted by identical or different alkyl radicals or aryl radicals, which are shown here in general terms by the radicals R₁-R₄ (that is to say the number of different radicals is not necessarily limited to 4). m can assume values from 2-200,000.

Cyclic silicones to be used advantageously according to the invention are generally characterized by structural elements, as follows 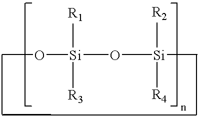
where the silicon atoms can be substituted by identical or different alkyl radicals or aryl radicals, which are shown here in general terms by the radicals R₁-R₄ (that is to say the number of different radicals is not necessarily limited to 4). n can assume values from 3/2 to 20. Fractions for n take into consideration that uneven numbers of siloxyl groups may be present in the cycle.

Advantageously, cyclomethicone (e.g. decamethylcyclopentasiloxane) is used as the silicone oil to be used according to the invention. However, other silicone oils are also to be used advantageously for the purpose of the present invention, for example undecamethylcyclotrisiloxane, polydimethylsiloxane, poly(methylphenylsiloxane), cetyldimethicone, behenoxydimethicone.

Also advantageous are mixtures of cyclomethicone and isotridecyl isononanoate, and those of cyclomethicone and 2-ethylhexyl isostearate.

It is, however, also advantageous to choose silicone oils of similar constitution to the above-described compounds whose organic side chains are derivatized, for example polyethoxylated or polypropoxylated. These include, for example, polysiloxane-polyalkyl-polyether copolymers, such as cetyl-dimethicone copolyol, (cetyl-dimethicone copolyol (and) polyglyceryl-4 isostearate (and) hexyl laurate).

Also particularly advantageous are mixtures of cyclomethicone and isotridecyl isononanoate, and of cyclomethicone and 2-ethylhexyl isostearate.

Preparations according to the invention in the form of emulsions comprise one or more emulsifiers. These emulsifiers can advantageously be chosen from the group of nonionic, anionic, cationic or amphoteric emulsifiers.

The nonionic emulsifiers include
a) partial fatty acid esters and fatty acid esters of polyhydric alcohols and ethoxylated derivatives thereof (e.g. glyceryl monostearates, sorbitan stearates, glyceryl stearyl citrates, sucrose stearates)
b) ethoxylated fatty alcohols and fatty acids
c) ethoxylated fatty amines, fatty acid amides, fatty acid alkanolamides
d) alkylphenol polyglycol ethers (e.g. Triton X).

The anionic emulsifiers include
a) soaps (e.g. sodium stearate)
b) fatty alcohol sulfates
c) mono-, di- and trialkyl phosphoic esters and ethoxylates thereof.
   The cationic emulsifiers include
a) quaternary ammonium compounds with a long-chain aliphatic radical, e.g. distearyidimonium chloride.
   The amphoteric emulsifiers include
   a) alkylamininoalkanecarboxylic acids
   b) betaines, sulfobetaines
   c) imidazoline derivatives.

In addition, there are naturally occurring emulsifiers, which include beeswax, wool wax, lecithin and sterols.

O/W emulsifiers can be advantageously chosen, for example, from the group of polyethoxylated or polypropoxylated or polyethoxylated and polypropoxylated products, e.g.:
fatty alcohol ethoxylates,
ethoxylated wool wax alcohols,
polyethylene glycol ethers of the general formula R—O—(—CH₂—CH₂—O—)ₙ—R′,
fatty acid ethoxylates of the general formula
   R—COO—(—CH₂—CH₂—O—)ₙ—H,
etherified fatty acid ethoxylates of the general formula
   R—COO—(—CH₂—CH₂—O—)ₙ—R′,
esterified fatty acid ethoxylates of the general formula
   R—COO—(—CH₂—CH₂—O—)ₙ—C(O)—R′,
polyethylene glycol glycerol fatty acid esters,
ethoxylated sorbitan esters,
cholesterol ethoxylates,
ethoxylated triglycerides,
alkyl ether carboxylic acids of the general formula
   R—O—(—CH₂—CH₂—O—)ₙ—CH₂—COOH
   and n are a number from 5 to 30,
polyoxyethylene sorbitol fatty acid esters,
alkyl ether sulfates of the general formula R-O-(—CH₂—CH₂—O—)ₙ—SO₃—H,
fatty alcohol propoxylates of the general formula
   R—O—(—CH₂—CH(CH₃)—O—)ₙ—H,
polypropylene glycol ethers of the general formula
   R—O—(—CH₂—CH(CH₃)—O—)ₙ—R′,
propoxylated wool wax alcohols,
etherified fatty acid propoxylates
   R—COO—(—CH₂—CH(CH₃)—O—)ₙ—R′,
esterified fatty acid propoxylates of the general formula
   R—COO—(—CH₂—CH(CH₃)—O—)ₙ—C(O)—R′,
fatty acid propoxylates of the general formula
   R—COO—(—CH₂—CH(CH₃)—O—)ₙ—H,
polypropylene glycol glycerol fatty acid esters,
propoxylated sorbitan esters,
cholesterol propoxylates,
propoxylated triglycerides,
alkyl ether carboxylic acids of the general formula
   R—O—(—CH₂—CH(CH₃)O—)ₙ—CH₂—COOH,
alkyl ether sulfates or the parent acids of these sulfates of the general formula
   R—O—(—CH₂—CH(CH₃)—O—)ₙ—SO₃—H,
fatty alcohol ethoxylates/propoxylates of the general formula
   R—O—Xₙ—Yₘ—H,
polypropylene glycol ethers of the general formula
   R—O—Xₙ—Yₘ—R′,
etherified fatty acid propoxylates of the general formula
   R—COO—Xₙ—Ym—R′,
fatty acid ethoxylates/propoxylates of the general formula
   R—COO—Xₙ—Yₘ—H.

According to the invention, particularly advantageous polyethoxylated or polypropoxylated or polyethoxylated and polypropoxylated O/W emulsifiers used are those chosen from the group of substances having HLB values of 11-18, very particularly preferably having HLB values of 14.5-15.5, provided the ONV emulsifiers have saturated radicals R and R′. If the O/N emulsifiers have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols (cetearyl alcohols). Particular preference is given to polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20), polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (isosteareth-16), polyethylene glycol(17) isostearyl ether (isosteareth-17), polyethylene glycol(18) isostearyl ether (isosteareth-18), polyethylene glycol(19) isostearyl ether (isosteareth-19), polyethylene glycol(20) isostearyl ether (isosteareth-20), polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol(20) cetyl ether (ceteth-20), polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), polyethylene glycol(18) isocetyl ether (isoceteth-18), polyethylene glycol(19) isocetyl ether (isoceteth-19), polyethylene glycol(20) isocetyl ether (isoceteth-20), polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15), polyethylene glycol(12) lauryl ether (laureth-12), polyethylene glycol(12) isolauryl ether (isolaureth-12), polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), and polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate, polyethylene glycol(12) isostearate, polyethylene glycol(13) isostearate, polyethylene glycol(14) isostearate, polyethylene glycol(15) isostearate, polyethylene glycol(16) isostearate, polyethylene glycol(17) isostearate, polyethylene glycol(18) isostearate, polyethylene glycol(19) isostearate, polyethylene glycol(20) isostearate, polyethylene glycol(21) isostearate, polyethylene glycol(22) isostearate, polyethylene glycol(23) isostearate, polyethylene glycol(24) isostearate, polyethylene glycol(25) isostearate, polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth 1-4-sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) evening primrose glycerides.

It is also advantageous to choose the polyethylene glycol glycerol fatty acid esters from the group polyethylene glycol(20) glyceryl laurate, polyethylene glycol(21) glyceryl laurate, polyethylene glycol(22) glyceryl laurate, polyethylene glycol(23) glyceryl laurate, polyethylene glycol(6) glyceryl caprate/caprinate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol(20) sorbitan monoisostearate, polyethylene glycol(20) sorbitan monopalmitate, polyethylene glycol(20) sorbitan monooleate.

Advantageous W/O emulsifiers which can be used are: fatty alcohols having 8 to 30 carbon atoms, monoglycerol esters of saturated and/or unsaturated, branched and/or unbranched alkanecarboxylic acids having a chain length of from 8 to 24, in particular 12-18, carbon atoms, diglycerol esters of saturated or unsaturated, branched or unbranched alkanecarboxylic acids having a chain length of from 8 to 24, in particular 12-18, carbon atoms, monoglycerol ethers of saturated or unsaturated, branched or unbranched alcohols having a chain length of from 8 to 24, in particular 12-18, carbon atoms, diglycerol ethers of saturated or unsaturated, branched or unbranched alcohols having a chain length of from 8 to 24, in particular 12-18, carbon atoms, propylene glycol esters of saturated or unsaturated, branched or unbranched alkanecarboxylic acids having a chain length of from 8 to 24, in particular 12-18, carbon atoms, and sorbitan esters of saturated or unsaturated, branched or unbranched alkanecarboxylic acids having a chain length of from 8 to 24, in particular 12-18, carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol(2) stearyl ether (steareth-2), glyceryl monolaurate, glyceryl monocaprinate, glyceryl monocaprylate.

It is also advantageous for the purposes of the present invention to create cosmetic and dermatological preparations whose main purpose is not protection against sunlight but which nevertheless have a content of further UV protection substances. Thus, for example, UV-A and/or UV-B filter substances are usually incorporated into day creams or makeup products. UV protection substances, like antioxidants and, if desired, preservatives, also represent effective protection of the preparations themselves against spoilage. Also favorable are cosmetic and dermatological preparations which are present in the form of a sunscreen composition.

Accordingly, the preparations for the purposes of the present invention preferably comprise at least one further UV-A, UV-B or broadband filter substance. The formulations may, although do not necessarily, optionally also comprise one or more organic and/or inorganic pigments as UV filter substances which may be present in the water phase and/or the oil phase.

In addition, the preparations according to the invention may advantageously also be present in the form of so-called oil-free cosmetic or dermatological emulsions which comprise a water phase and at least one UV filter substance which is liquid at room temperature as a further phase.

Particularly advantageous UV filter substances which are liquid at room temperature for the purposes of the present invention are homomenthyl salicylate (INCI: Homosalate), 2-ethylhexyl 2-cyano-3,3-diphenylacrylate (INCI: Octocrylene), 2-ethylhexyl 2-hydroxybenzoate (2-ethylhexyl salicylate, octyl salicylate, INCI: Octyl Salicylate) and esters of cinnamic acid, preferably 2-ethylhexyl 4-methoxycinnamate (INCI: Octyl Methoxycinnamate) and isopentyl 4-methoxycinnamate (INCI: Isoamyl p-Methoxycinnamate).

Preferred inorganic pigments are metal oxides and/or other metal compounds which are insoluble or sparingly soluble in water, in particular oxides of titanium (TiO₂), zinc (ZnO), iron (e.g. Fe₂O₃), zirconium (ZrO₂), silicon (SiO₂), manganese (e.g. MnO), aluminum (Al₂O₃), cerium (e.g. Ce₂O₃), mixed oxides of the corresponding metals, and mixtures of such oxides, and the sulfate of barium (BaSO₄).

For the purposes of the present invention, the pigments may advantageously also be used in the form of commercially available oily or aqueous predispersions. Dispersion auxiliaries and/or solubilization promoters may advantageously be added to these predispersions.

According to the invention, the pigments can advantageously be surface-treated (“coated”), the intention being, for example, to form or retain a hydrophilic, amphiphilic or hydrophobic character. This surface treatment can consist in providing the pigments with a thin hydrophilic or hydrophobic inorganic or organic layer by methods known per se. For the purposes of the present invention, the various surface coatings may also comprise water.

Inorganic surface coatings for the purposes of the present invention may consist of aluminum oxide (Al₂O₃), aluminum hydroxide Al(OH)₃, or aluminum oxide hydrate (also: alumina, CAS No.: 1333-84-2), sodium hexamethaphosphate (NaPO₃)₆, sodium metaphosphate (NaPO₃)ₙ, silicon dioxide (SiO₂) (also: silica, CAS No.: 7631-86-9), or iron oxide (Fe₂O₃). These inorganic surface coatings may be present on their own, in combination, or in combination with organic coating materials.

Organic surface coatings for the purposes of the present invention may consist of vegetable or animal aluminum stearate, vegetable or animal stearic acid, lauric acid, dimethylpolysiloxane (also: dimethicone), methylpolysiloxane (methicone), simethicones (a mixture of dimethylpolysiloxane with an average chain length of from 200 to 350 dimethylsiloxane units and silica gel) or alginic acid. These organic surface coatings can be used on their own, in combination and/or in combination with inorganic coating materials.

Zinc oxide particles and predispersions of zinc oxide particles which are suitable according to the invention are available under the following trade names from the companies listed:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |

Suitable titanium dioxide particles and predispersions of titanium dioxide particles are available under the following trade names from the companies listed:

| Trade name | Coating | Manufacturer |
|---|---|---|
| MT-100TV | Aluminum hydroxide/stearic | Tayca Corporation |
| | acid | |
| MT-100Z | Aluminum hydroxide/stearic | Tayca Corporation |
| | acid | |
| Eusolex T-2000 | Alumina/simethicone | Merck KgaA |
| Titandioxid T805 | Octyltrimethylsilane | Degussa |
| (Uvinul TiO₂) | | |

Advantageous UV-A filter substances for the purposes of the present invention are dibenzoylmethane derivatives, in particular 4-(tert-butyl)-4′-methoxydibenzoylmethane (CAS No. 70356-09-1), which is sold by Givaudan under the name Parsol® 1789 and by Merck under the trade name Eusolex®9020.

Advantageous further UV filter substances for the purposes of the present invention are sulfonated, water-soluble UV filters, such as, for example:
phenylene-1,4-bis(2-benzimidazyl)-3,3′-5,5′-tetrasulfonic acid and its salts, particularly the corresponding sodium, potassium or triethanolammonium salts, in particular the phenylene-1,4-bis(2-benzimidazyl)-3,3′-5,5′-tetrasulfonic acid bis-sodium salt with the INCI name Bisimidazylate (CAS No.: 180898-37-7), which is available, for example, under the trade name Neo Heliopan AP from Haarmann & Reimer;
salts of 2-phenylbenzimidazol-5-sulfonic acid, such as its sodium, potassium or its triethanolammonium salt, and the sulfonic acid itself with the INCI name Phenylbenzimidazole Sulfonic Acid (CAS No.: 27503-81-7), which is available, for example, under the trade name Eusolex 232 from Merck or under Neo Heliopan Hydro from Haarmann & Reimer;
1,4-di(2-oxo-10-sulfo-3-bornylidenemethyl)benzene (also: 3,3′-(1,4-phenylene-dimethylene)bis(7,7-dimethyl-2-oxobicyclo[2.2.1]hept-1-ylmethanesulfonic acid) and salts thereof (preferably the corresponding 10-sulfato compounds, in particular the corresponding sodium, potassium or triethanolammonium salt), which is also referred to as benzene-1,4-di(2-oxo-3-bornylidenemethyl-10-sulfonic acid). Benzene-1,4-di(2-oxo-3-bornylidenemethyl-10-sulfonic acid) has the INCI name Terephthalidene Dicamphor Sulfonic Acid (CAS No.: 90457-82-2) and is available, for example, under the trade name Mexoryl SX from Chimex;
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)sulfonic acid and salts thereof.

Advantageous UV filter substances for the purposes of the present invention are also so-called broadband filters, i.e. filter substances which absorb both UV-A and UV-B radiation.

Advantageous broadband filters or UV-B filter substances are, for example, triazine derivatives, such as, for example,
2,4-bis{[4-(2-ethylhexyloxy)-2-hydroxylphenyl}-6-(4-methoxyphenyl)-1,3,5-triazine (INCI: Aniso Triazine), which is available under the trade name Tinosorb® S from CIBA-Chemikalien GmbH;
diethylhexylbutylamidotriazone (INCI: Diethylhexylbutamidotriazone), which is available under the trade name UVASORB HEB from Sigma 3V;
tris(2-ethylhexyl) 4,4′,4″-(1,3,5-triazine-2,4,6-triyltriimino)trisbenzoate, also: 2,4,6-tris[anilino(p-carbo-2′-ethyl-1′-hexyloxy)]-1,3,5-triazine (INCI: Ethylhexyl Triazone), which is sold by BASF Aktiengesellschaft under the trade name UVINUL® T 150.

An advantageous broadband filter for the purposes of the present invention is also 2,2′-methylene-bis(6-(2H-benzotriazol-2-yl)-4-(1,1,3,3-tetramethylbutyl) phenol), which is available under the trade name Tinosorb® M from CIBA-Chemikalien GmbH.

An advantageous broadband filter for the purposes of the present invention is also 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane, which is available under the trade name Mexoryl® XL from Chimex.

The further UV filter substances may be oil-soluble or water-soluble.

Advantageous oil-soluble UV-B and/or broadband filter substances for the purposes of the present invention are, for example:
3-benzylidenecamphor derivatives, preferably 3-(4-methylbenzylidene)-camphor, 3-benzylidenecamphor;
4-aminobenzoic acid derivatives, preferably 2-ethylhexyl 4-(dimethylamino)benzoate, amyl 4-(dimethylamino)benzoate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone
and UV filters bonded to polymers
3-(4-(2,2-bisethoxycarbonylvinyl)phenoxy)propenyl)methoxysiloxane/dimethylsiloxane copolymer which is available, for example, under the trade name Parsol® SLX from Hoffmann La Roche.

Advantageous water-soluble filter substances are, for example:

sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and salts thereof.

A further light protection filter substance to be used advantageously according to the invention is ethylhexyl 2-cyano-3,3-diphenylacrylate (octocrylene), which is available from BASF under the name Uvinul® N 539.

Particularly advantageous preparations for the purposes of the present invention which are characterized by high or very high UV-A and/or UV-B protection preferably also comprise further UV-A and/or broadband filters, in particular dibenzoylmethane derivatives [for example 4-(tert-butyl)-4′-methoxydibenzoylmethane], phenylene-1,4-bis(2-benzimidazyl)-3,3′-5,5′-tetrasulfonic acid and its salts, 1,4-di(2-oxo-10-sulfo-3-bornylidenemethyl)benzene and salts thereof and 2,4-bis{[4-(2-ethylhexyloxy)-2-hydroxylphenyl}-6-(4-methoxyphenyl)-1,3,5-triazine, in each case individually or in any combinations with one another, besides the filter substance(s) according to the invention.

The list of specified UV filters which can be used for the purposes of the present invention is not of course intended to be limiting.

Advantageously, the preparations according to the invention comprise the substances which absorb UV radiation in the UV-A and/or UV-B region in a total amount of, for example, 0.1% by weight to 30% by weight, preferably 0.5 to 20%, in particular 1.0% to 15.0% by weight, in each case based on the total weight of the preparations, in order to provide cosmetic preparations which protect the hair and/or the skin from the entire range of ultraviolet radiation.

Particularly advantageous preparations are also obtained when antioxidants are used as additives or active ingredients. According to the invention, the preparations advantageously comprise one or more antioxidants. Favorable, but nevertheless optional, antioxidants which may be used are all antioxidants which are customary or suitable for cosmetic and/or dermatological application.

It is also advantageous to add antioxidants to the preparations according to the invention. The antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine for example, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxyfatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. y-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate), and coniferyl benzoate of the benzoin resin, rutinic acid and derivatives thereof, α-glycosylrutin, ferulic acid, furfurylideneglucitol, carnosine, butylhydroxytoluene, butylhydroxyanisole, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives of these specified active ingredients which are suitable according to the invention (salts, esters, ethers, sugars, nucleotides, nucleosides, peptides and lipids).

Oil-soluble antioxidants can be used particularly advantageously for the purposes of the present invention.

A surprising property of the present invention is that preparations according to the invention are very good vehicles for cosmetic or dermatological active ingredients into the skin, preferred active ingredients being antioxidants which can protect the skin against oxidative stress. Preferred antioxidants here are vitamin E and derivatives thereof, and vitamin A and derivatives thereof.

The amount of antioxidants (one or more compounds) in the preparations is preferably 0.001 to 30% by weight, particularly preferably 0.05-20% by weight, in particular 1-10% by weight, based on the total weight of the preparation.

If vitamin E and/or derivatives thereof are the antioxidant or the antioxidants, it is advantageous to chose their particular concentrations from the range 0.001-10% by weight, based on the total weight of the formulation.

If vitamin A, or vitamin A derivatives, or carotenes or derivatives thereof are the antioxidant or the antioxidants, it is advantageous to chose their particular concentrations from the range 0.001-10% by weight, based on the total weight of the formulation.

The examples below are intended to illustrate the present invention without limiting it. The numerical values in the examples are percentages by weight, based on the total weight of the particular preparations.

### EXAMPLES

1. O/W creams

| | 1 | 2 | 3 | 4 | 5 |
|---|---|---|---|---|---|
| Glyceryl stearate citrate | | | 2.00 | | 2.00 |
| Glyceryl stearate, self emulsifying | 4.00 | 3.00 | | | |
| PEG-40 stearate | 1.00 | | | | |
| Polyglyceryl-3 methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetyl stearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4- | | | 0.50 | | |
| methoxyphenyl)-1,3-propanedione | | | | | |
| 8-Hydroxyguanosine | 0.0001 | 0.0001 | 0.0001 | 0.00001 | 0.00001 |
| Tocopherol | 0.1 | | | | 0.20 |
| Biotin | | | 0.05 | | |
| Ethylenediaminetetraacetic | 0.1 | | 0.10 | 0.1 | |
| acid trisodium | | | | | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |

2. PIT emulsions

| | 1 | 2 | 3 | 4 | 5 |
|---|---|---|---|---|---|
| Glycerol monostearate, self-emulsifying | 0.50 | | 3.00 | 2.00 | 4.00 |
| Polyoxyethylene(12) cetylstearyl ether | | 5.00 | | 1.00 | 1.50 |
| Polyoxyethylene(20) cetylstearyl ether | | | | 2.00 | |
| Polyoxyethylene(30) cetylstearyl ether | 5.00 | | 1.00 | | |
| Stearyl alcohol | | | 3.00 | | 0.50 |
| Cetyl alcohol | 2.50 | 1.00 | | 1.50 | |
| 2-Ethylhexyl methoxycinnamate | | | | 5.00 | 8.00 |
| 2,4-Bis(4-(2-ethylhexyloxy)-2-hydroxy)- | | 1.50 | | 2.00 | 2.50 |
| phenyl)-6-(4- | | | | | |
| methoxyphenyl)(1,3,5)triazine | | | | | |
| 1-(4-tert-Butylphenyl)-3-(4- | | | 2.00 | | |
| methoxyphenyl)-1,3-propanedione | | | | | |
| Diethylhexylbutamidotriazone | 1.00 | 2.00 | | 2.00 | |
| Ethylhexyltriazone | 4.00 | | 3.00 | 4.00 | |
| 4-Methylbenzylidenecamphor | | 4.00 | | | 2.00 |
| Octocrylene | | 4.00 | | | 2.50 |
| Phenylene-1,4-bis(monosodium, | | | 0.50 | | 1.50 |
| 2-benzimidazyl-5,7-disulfonic acid) | | | | | |
| Phenylbenzimidazolesulfonic acid | 0.50 | | | 3.00 | |
| C12-15 Alkyl benzoate | | 2.50 | | | 5.00 |
| Titanium dioxide | 0.50 | 1.00 | | 3.00 | 2.00 |
| Zinc oxide | 2.00 | | 3.00 | 0.50 | 1.00 |
| Dicaprylyl ether | | | 3.50 | | |
| Butylene glycol dicaprylate/dicaprate | 5.00 | | | 6.00 | |
| Dicaprylyl carbonate | | | 6.00 | | 2.00 |
| Dimethicone polydimethylsiloxane | | 0.50 | 1.00 | | |
| Phenylmethylpolysiloxane | 2.00 | | | 0.50 | 0.50 |
| Shea butter | | 2.00 | | | 0.50 |
| PVP hexadecene copolymer | 0.50 | | | 0.50 | 1.00 |
| Glycerol | 3.00 | 7.50 | 5.00 | 7.50 | 2.50 |
| Tocopherol acetate | 0.50 | | 0.25 | | 1.00 |
| 8-Hydroxyguanosine | 0.0001 | 0.0001 | 0.0001 | 0.00001 | 0.00001 |
| alpha-Glucosylrutin | 0.10 | | 0.20 | | |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

3. O/W Creams

| Examples | 1 | 2 | 3 | 4 | 5 |
|---|---|---|---|---|---|
| Glyceryl stearate citrate | | | 2.00 | | 2.00 |
| Glyceryl stearate, self emulsifying | 4.00 | 3.00 | | | |
| PEG-40 stearate | 1.00 | | | | |
| Polyglyceryl-3 methylglucose distearate | | | | 3.00 | |
| Sorbitan stearate | | | | | 2.00 |
| Stearic acid | | 1.00 | | | |
| Stearyl alcohol | | | 5.00 | | |
| Cetyl alcohol | 3.00 | 2.00 | | 3.00 | |
| Cetylstearyl alcohol | | | | | 2.00 |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidenecamphor | | | 1.00 | | |
| 1-(4-tert-Butylphenyl)-3-(4- | | | 0.50 | | |
| methoxyphenyl)-1,3-propanedione | | | | | |
| 8-Hydroxyguanosine | 0.0001 | 0.0001 | 0.0001 | 0.00001 | 0.00001 |
| Tocopherol | 0.1 | | | | 0.20 |
| Biotin | | | 0.05 | | |
| Ethylenediaminetetraacetic acid | 0.1 | | 0.10 | 0.1 | |
| trisodium | | | | | |
| Preservative | q.s. | q.s. | q.s. | q.s. | q.s. |
| Polyacrylic acid | 3.00 | 0.1 | | 0.1 | 0.1 |
| Sodium hydroxide solution 45% | q.s. | q.s. | q.s. | q.s. | q.s. |
| Glycerol | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |

4. W/O Emulsions

| | 1 | 2 | 3 | 4 | 5 |
|---|---|---|---|---|---|
| Cetyldimethicone copolyol | | 2.50 | | 4.00 | |
| Polyglyceryl-2 dipolyhydroxystearate | 5.00 | | | | 4.50 |
| PEG-30 dipolyhydroxystearate | | | 5.00 | | |
| 2-Ethylhexyl methoxycinnamate | | 8.00 | | 5.00 | 4.00 |
| 2,4-Bis(4-(2-ethylhexyloxy)-2-hydroxy)- | 2.00 | 2.50 | | 2.00 | 2.50 |
| phenyl)-6-(4- | | | | | |
| methoxyphenyl)(1,3,5)triazine | | | | | |
| 1-(4-tert-Butylphenyl)-3-(4- | | | 2.00 | 1.00 | |
| methoxyphenyl)-1,3-propanedione | | | | | |
| Diethylhexylbutamidotriazone | 3.00 | 1.00 | | | 3.00 |
| Ethylhexyltriazone | | | 3.00 | 4.00 | |
| 4-Methylbenzylidenecamphor | | 2.00 | | 4.00 | 2.00 |
| Octocrylene | 7.00 | 2.50 | 4.00 | | 2.50 |
| Diethylhexylbutamidotriazone | 1.00 | | | 2.00 | |
| Phenylene-1,4-bis(monosodium, | 1.00 | 2.00 | 0.50 | | |
| 2-benzimidazyl-5,7-disulfonic acid) | | | | | |
| Phenylbenzimidazolesulfonic acid | 0.50 | | | 3.00 | 2.00 |
| Titanium dioxide | | 2.00 | 1.50 | | 2.00 |
| Zinc oxide | 3.00 | 1.00 | 2.00 | 0.50 | |
| Paraffinum liquidum | | | 10.00 | | 8.00 |
| C12-C15 Alkyl benzoate | | | | 9.00 | |
| Dicaprylyl ether | 10.00 | | | | 7.00 |
| Butylene glycol dicaprylate/dicaprate | | | 2.00 | 8.00 | 4.00 |
| Dicaprylyl carbonate | 5.00 | | 6.00 | | |
| Dimethicone polydimethylsiloxane | | 4.00 | 1.00 | 5.00 | |
| Phenylmethylpolysiloxane | 2.00 | 25.00 | | | 2.00 |
| Shea butter | | | 3.00 | | |
| PVP hexadecene copolymer | 0.50 | | | 0.50 | 1.00 |
| Octoxyglycerol | | 0.30 | 1.00 | | 0.50 |
| Glycerol | 3.00 | 7.50 | | 7.50 | 2.50 |
| Glycine soya | | 1.00 | 1.50 | | |
| Magnesium sulfate | 1.00 | 0.50 | | 0.50 | |
| Magnesium chloride | | | 1.00 | | 0.70 |
| Tocopherol acetate | 0.50 | | 0.25 | | 1.00 |
| 8-Hydroxyguanosine | 0.0001 | 0.0001 | 0.0001 | 0.00001 | 0.00001 |
| Preservative | q.s. | q.s. | q.s. | q.s. | q.s. |
| Ethanol | 3.00 | | 1.50 | | 1.00 |
| Perfume | q.s. | q.s. | q.s. | q.s. | q.s. |
| Water | ad 100 | ad 100 | ad 100 | ad 100 | ad 100 |

5. Hydrodispersions

| | 1 | 2 | 3 | 4 | 5 |
|---|---|---|---|---|---|
| Polyoxyethylene(20) cetylstearyl ether | 1.00 | | | 0.5 | |
| Cetyl alcohol | | | 1.00 | | |
| Sodium polyacrylate | | 0.20 | | 0.30 | |
| Acrylates/C10-30-alkyl acrylate | 0.50 | | 0.40 | 0.10 | 0.10 |
| crosspolymer | | | | | |
| Xanthan gum | | 0.30 | 0.15 | | 0.50 |
| 2-Ethylhexyl methoxycinnamate | | | | 5.00 | 8.00 |
| 2,4-Bis(4-(2-ethylhexyloxy)-2-hydroxy)- | | 1.50 | | 2.00 | 2.50 |
| phenyl)-6-(4- | | | | | |
| methoxyphenyl)(1,3,5)triazine | | | | | |
| 1-(4-tert-Butylphenyl)-3-(4- | 1.00 | | 2.00 | | |
| methoxyphenyl)-1,3-propanedione | | | | | |
| Diethylhexylbutamidotriazone | | 2.00 | | 2.00 | 1.00 |
| Ethylhexyltriazone | 4.00 | | 3.00 | 4.00 | |
| 4-Methylbenzylidenecamphor | 4.00 | 4.00 | | | 2.00 |
| Octocrylene | | 4.00 | 4.00 | | 2.50 |
| Phenylene-1,4-bis(monosodium, | 1.00 | | 0.50 | | 2.00 |
| 2-benzimidazyl-5,7-disulfonic acid) | | | | | |
| Phenylbenzimidazolesulfonic acid | 0.50 | | | 3.00 | |
| Titanium dioxide | 0.50 | | 2.00 | 3.00 | 1.00 |
| Zinc oxide | 0.50 | 1.00 | 3.00 | | 2.00 |
| C12-C15 Alkyl benzoate | 2.00 | 2.50 | | | |
| Dicaprylyl ether | | 4.00 | | | 7.00 |
| Butylene glycol dicaprylate/dicaprate | 4.00 | | 2.00 | 6.00 | |
| Dicaprylyl carbonate | | 2.00 | 6.00 | | |
| Dimethicone polydimethylsiloxane | | 0.50 | 1.00 | | |
| Phenylmethylpolysiloxane | 2.00 | | | 0.50 | 2.00 |
| Shea butter | | 2.00 | | | |
| PVP hexadecene copolymer | 0.50 | | | 0.50 | 1.00 |
| Octoxyglycerol | | | 1.00 | | 0.50 |
| Glycerol | 3.00 | 7.50 | | 7.50 | 2.50 |
| Glycine soya | | | 1.50 | | |
| Tocopherol acetate | 0.50 | | 0.25 | | 1.00 |
| 8-Hydroxyguanosine | 0.0001 | 0.0001 | 0.0001 | 0.00001 | 0.00001 |
| Preservative | q.s. | q.s. | q.s. | q.s. | q.s. |
| Ethanol | 3.00 | 2.00 | 1.50 | | 1.00 |
| Perfume | q.s. | q.s. | q.s. | q.s. | q.s. |
| Water | ad 100 | ad 100 | ad 100 | ad 100 | ad 100 |

6. Gel Cream

| | |
|---|---|
| Acrylate/C10-30 alkyl acrylate crosspolymer | 0.40 |
| Polyacrylic acid | 0.20 |
| Xanthan gum | 0.10 |
| Cetearyl alcohol | 3.00 |
| C12-15 Alkyl benzoate | 4.00 |
| Caprylic/capric triglyceride | 3.00 |
| Cyclic dimethylpolysiloxane | 5.00 |
| Dimethicone polydimethylsiloxane | 1.00 |
| 8-Hydroxyguanosine | 0.0001 |
| Glycerol | 3.00 |
| Sodium hydroxide | q.s. |
| Preservative | q.s. |
| Perfume | q.s. |
| Water | ad 100.0 |
| pH adjusted to 6.0 | |

7. W/O Cream

| | |
|---|---|
| Polyglyceryl-3 diisostearate | 3.50 |
| Glycerol | 3.00 |
| Polyglycerol-2 dipolyhydroxystearate | 3.50 |
| 8-Hydroxyguanosine | 0.0001 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

8. W/O/W Cream

| | |
|---|---|
| Glyceryl stearate | 3.00 |
| PEG-100 stearate | 0.75 |
| Behenyl alcohol | 2.00 |
| Caprylic/capric triglyceride | 8.0 |
| Octydodecanol | 5.00 |
| C12-15 Alkyl benzoate | 3.00 |
| 8-Hydroxyguanosine | 0.0001 |
| Magnesium sulfate (MgSO₄) | 0.80 |
| Ethylenediaminetetraacetic acid | 0.10 |
| Preservative | q.s. |
| Perfume | q.s. |
| Water | ad 100.0 |
| pH adjusted to 6.0 | |

1. A cosmetic or dermatological preparation comprising at least one reaction product of DNA selected from the group consisting of oxidation products of mononucleosides, oxidation products of nucleobases, photoproducts of mononucleosides, and photoproducts of nucleobases.

2. The cosmetic or dermatological preparation as claimed in claim 1, wherein the at least one reaction product of DNA includes at least one reaction product selected from the group consisting of oxidation products of mononucleosides and photoproducts of nucleobases.

3. The cosmetic or dermatological preparation as claimed in claim 2, wherein the at least one reaction product of DNA includes at least one pyrimidine-6,4 photoproduct.

4. The cosmetic or dermatological preparation as claimed in claim 3, wherein the pyrimidine-6,4 photoproduct has the chemical structure 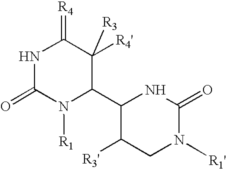
wherein R₃ and R₃′ are independently of one another selected from the group consisting of methyl or hydrogen, and wherein R₄ is selected from the group consisting of oxygen and NH in an imido group, and R₄′ is selected from the group consisting of amino and hydroxyl groups.

5. The cosmetic or dermatological preparation as claimed in claim 1, wherein the at least one reaction product of DNA includes at least one thymidine photodimer formed from DNA by photo-induced {2+2]-cycloaddition having the chemical structure 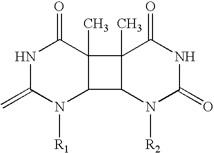
wherein R₁ and R₂ are independently of one another selected from the group consisting of carbohydrates, or carbohydrate derivatives thereof, and nucleotide chains.

6. The cosmetic or dermatological preparation as claimed in claim 1, wherein the concentration of the at least one reaction product of DNA is from 0.000001 to 0.1% by weight, based on the total weight of the preparation.

7. The cosmetic or dermatological preparation as claimed in claim 6, wherein the concentration of the at least one reaction product of DNA is from 0.000001 to 0.001% by weight, based on the total weight of the preparation.

8. The cosmetic or dermatological preparation as claimed in claim 1, wherein the preparation is an emulsion.

9. The cosmetic or dermatological preparation as claimed in claim 1, further comprising at least one antioxidant.

10. The cosmetic or dermatological preparation as claimed in claim 9, wherein the at least one antioxidant includes at least one antioxidant selected from the group consisting of vitamin E, vitamin E derivatives, vitamin A, and vitamin A derivatives.

11. A cosmetic or dermatological preparation comprising at least one 8-hydroxypurine nucleoside.

12. The cosmetic or dermatological preparation as claimed in claim 11, wherein the 8-hydroxypurine nucleoside is 8-hydroxyguanosine.

13. The cosmetic or dermatological preparation as claimed in claim 11, wherein the concentration of the at least one 8-hydroxypurine nucleoside is from 0.000001 to 0.1% by weight, based on the total weight of the preparation.

14. The cosmetic or dermatological preparation as claimed in claim 11, wherein the concentration of the at least one 8-hydroxypurine nucleoside is from 0.000001 to 0.001% by weight, based on the total weight of the preparation.

15. The cosmetic or dermatological preparation as claimed in claim 11, wherein the preparation is an emulsion.

16. The cosmetic or dermatological preparation as claimed in claim 11, further comprising at least one antioxidant.

17. The cosmetic or dermatological preparation as claimed in claim 16, wherein the at least one antioxidant includes at least one antioxidant selected from the group consisting of vitamin E, vitamin E derivatives, vitamin A, and vitamin A derivatives.

18. A method of preventing or treating degenerative or inflammatory skin symptoms comprising applying to the skin a cosmetic or dermatological preparation comprising at least one reaction product of DNA selected from the group consisting of oxidation products of mononucleosides, oxidation products of nucleobases, photoproducts of mononucleosides, and photoproducts of nucleobases.

19. The method as claimed in claim 18, wherein the at least one reaction product of DNA includes at least one pyrimidine-6,4 photoproduct.

20. The method as claimed in claim 19, wherein the pyrimidine-6,4 photoproduct has the chemical structure 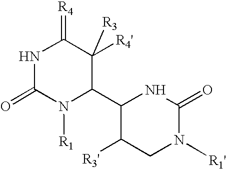
wherein R₃ and R₃′ are independently of one another selected from the group consisting of methyl or hydrogen, and wherein R₄ is selected from the group consisting of oxygen and NH in an imido group, and R₄′ is selected from the group consisting of amino and hydroxyl groups.

21. The method as claimed in claim 18, wherein the at least one reaction product of DNA includes at least one thymidine photodimer formed from DNA by photo-induced {2+2]-cycloaddition having the chemical structure 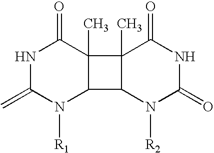
wherein R₁ and R₂ are independently of one another selected from the group consisting of carbohydrates, or carbohydrate derivatives thereof, and nucleotide chains.

22. The method as claimed in claim 18, wherein the at least one reaction product of DNA includes at least one 8-hydroxypurine nucleoside.

23. The method as claimed in claim 22, wherein the 8-hydroxypurine nucleoside is 8-hydroxyguanosine.

24. A method of reducing skin wrinkles, preventing skin aging or firming the skin comprising applying to the skin a cosmetic or dermatological preparation comprising at least one reaction product of DNA selected from the group consisting of oxidation products of mononucleosides, oxidation products of nucleobases, photoproducts of mononucleosides, and photoproducts of nucleobases.

25. The method as claimed in claim 24, wherein the at least one reaction product of DNA includes at least one pyrimidine-6,4 photoproduct.

26. The method as claimed in claim 25, wherein the pyrimidine-6,4 photoproduct has the chemical structure 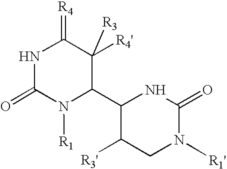
wherein R₃ and R₃′ are independently of one another selected from the group consisting of methyl or hydrogen, and wherein R₄ is selected from the group consisting of oxygen and NH in an imido group, and R₄′ is selected from the group consisting of amino and hydroxyl groups.

27. The method as claimed in claim 24, wherein the at least one reaction product of DNA includes at least one thymidine photodimer formed from DNA by photo-induced {2+2]-cycloaddition having the chemical structure 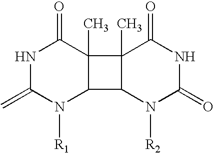
wherein R₁ and R₂ are independently of one another selected from the group consisting of carbohydrates, or carbohydrate derivatives thereof, and nucleotide chains.

28. The method as claimed in claim 24, wherein the at least one reaction product of DNA includes at least one 8-hydroxypurine nucleoside.

29. The method as claimed in claim 28, wherein the 8-hydroxypurine nucleoside is 8-hydroxyguanosine.

30. A method of treating the skin comprising
(a) applying to the skin a cosmetic or dermatological preparation comprising at least one reaction product of DNA selected from the group consisting of oxidation products of mononucleosides, oxidation products of nucleobases, photoproducts of mononucleosides, and photoproducts of nucleobases and
(b) topically applying a laser or abrasive treatment to the skin,
wherein (a) is performed either before, after, or both before and after (b).
